(19) United States
(12) Patent Application Publication
(10) Pub. No.: US 2006/0275516 A1
(43) Pub. Date: Dec. 7, 2006

(54) Compositions and methods for the treatment of allergic rhinitis

(76) Inventors: A.N. Sunder Ram, San Jose, CA (US); Bhupendra O. Shah, San Ramon, CA (US)

Correspondence Address: Sharon R. Kantor;c/o: The Firenza Group Ltd., 65 Panorama Court, Danville, CA, 94506-6154, US

(21) Appl. No.: 11/336,534

(22) Filed: Jan. 20, 2006

Related U.S. Application Data
(60) Provisional application No. 60/687,186, filed on Jun. 2, 2005.

(51) Int. Cl. A61K 36/906; A61K 36/28; A61K 31/12

(52) U.S. Cl. 424/756; 424/764; 514/690; 514/678

(57) ABSTRACT

The invention concerns novel pharmaceutical compositions and methods for the treatment of allergic rhinitis. More specifically, the invention concerns the inhibition of histamines, which is one of the primary causes of allergic rhinitis in human patients. The inventive pharmaceutical compositions include a carbonyl group-containing moiety, optionally in acidic medium, that combines with histamine to block histamine activity and thus reduce undesirable physiological effects of histamine in the body.

### BACKGROUND OF THE INVENTION

1. Field of the Invention

The present invention relates to the treatment of allergic rhinitis in general, and more specifically, to a class of compounds that inhibit allergy-causing histamine and methods for their use.

2. Description of the Related Art

Allergic rhinitis is the most common allergic disease in the United States and it affects an estimated 40 million people annually. Its prevalence and negative impact on quality of life make it an important health issue. Moreover, it costs two million lost workdays and three million lost school days due to the symptoms of the disease and/or side effects of the medications used to treat them. Allergic rhinitis can often contribute to other illnesses such as asthma and rhinosinusitis. There is mounting evidence that allergic diseases including rhinitis have been on the rise over the past decades.

Allergic rhinitis is a hypersensitivity response to specific allergens in sensitized people that act on a specific protein in the blood called immunoglobin E (IgE) antibody. Sensitized subjects with allergic rhinitis have IgE antibodies for specific allergen(s) bound to receptors on the surface of mast cells. On re-exposure to the specific allergen(s), cross-linking of adjacent IgE molecules occurs, and mast cell degranulation takes place resulting in the release of a variety chemical mediators. It is these mediators that cause the symptoms of allergic rhinitis. The major constituent and the best known mediator is histamine, but there are others, such as leukotrienes and prostaglandins all of which cause nasal allergic symptoms. Histamine, an inflammatory chemical, attaches to histamine receptors in blood vessels located in nasal tissues leading to a cascade of events that may cause symptoms of allergic rhinitis, including congestion, itching, sneezing, and rhinorrhea, also known as runny nose.

There are several over-the-counter (OTC) and prescription medications commonly available to treat allergic rhinitis. These include antihistamines, mast cell stabilizers, decongestants, combination medicines and corticosteroids. Each type does have benefits as well as side effects to consider.

Allergic rhinitis is frequently treated by a class of medications called antihistamines. Antihistamines act by blocking histamine receptors and not by binding histamine itself. Antihistamines can be either sedating or non-sedating in nature. Many over-the-counter antihistamines cause drowsiness, which can make driving or operating heavy machinery dangerous. According to results of a survey conducted among members of the National Association of School Nurses (NASN), 75 percent of school nurses polled agree that the physical side effects of the medications used to treat nasal allergy symptoms interfered with school performance, with 67 percent saying a child's alertness and ability to concentrate are impacted. In addition, 68 percent say that drowsiness has the greatest impact on children's participation both in and out of the classroom.

Mast cell stabilizers are another type of treatment for allergic rhinitis, the most well known of which is cromolyn sodium. These compounds inhibit the release of histamine from mast cells. In general, they are not considered to be as efficacious as inhaled corticosteroids, and suffer from the disadvantage that they are known to cause lightheadedness and headaches in some patients.

Decongestants are used to reduce nasal congestion, but may have little or no effect on itching, sneezing, or rhinorrhea. They can be effective when combined with an antihistamine. Nasal spray decongestants used for more than three days may actually make symptoms worse due to the rebound effect. For example, decongestants may cause the narrowing or constriction of blood vessels, also known as vasoconstriction, which can slow or restrict the flow of blood.

Corticosteroids are one class of potent allergy medications. These prescription drugs are used to reduce inflammation and other nasal allergy symptoms. However, they may not take full effect until they have been used for one to two weeks.

Current treatments for allergic rhinitis may thus be categorized according to the following mechanisms of action:
1. Anti-allergy drugs that inhibit the effects of histamine via blockage of its receptor, an example of which are the currently available antihistamines;
2. Anti-allergy drugs that prevent mast cells from releasing histamine by stabilizing the mast cell membrane;
3. Anti-allergy drugs that are decongestants that reverse one of the effects of histamine via vasoconstriction; and
4. Anti-allergy drugs that prevent or reduce inflammation caused by an allergic reaction, such as corticosteroids.

The foregoing techniques may be used to prevent and/or treat allergic rhinitis by interfering with the underlying causes of inflammation or by reversing the effects of inflammation. However, when considering a pharmacologic approach to treatment, it is important to consider both the efficacy and safety profiles of the various available medications.

### SUMMARY OF THE INVENTION

Compounds capable of blocking histamine with less adverse effects are always desirable. The present invention describes a method and compositions for treating allergic rhinitis. In one aspect, the invention concerns the blocking of binding sites on histamine. In another aspect, the compositions of the present invention involve the reversible condensation reaction of aldehydes and ketones with amines to give imines or enamines. Specifically, a carbonyl containing compound can react with histamine to block histamine activity and thus reduce undesirable physiological effects of histamine in the body. In yet another aspect of the present invention, a carbonyl containing compound can form an imine, an enamine or a combination of the foregoing upon reaction with an amine in acidic medium which, when hydrolyzed in the body, can release the carbonyl compound to react with and therefore block histamine in situ.

### BRIEF DESCRIPTION OF THE DRAWINGS

FIG. 1 is a Fourier Transform Infrared (FT IR) spectrum of a sample of curcumin;

FIG. 2 is a Fourier Transform Infrared (FT IR) spectrum of a sample of histamine;

FIG. 3 is a Fourier Transform Infrared (FT IR) spectrum of a first mixture of curcumin and histamine;

FIG. 4 is a Fourier Transform Infrared (FT IR) spectrum of a second mixture of curcumin and histamine in acid medium;

FIG. 5 is a Fourier Transform Infrared (FT IR) spectrum of a second mixture of curcumin;

FIG. 6 is a Fourier Transform Infrared (FT IR) spectrum of a third mixture of curcumin and histamine; and

FIG. 7 is a Fourier Transform Infrared (FT IR) spectrum of a fourth mixture of curcumin and histamine in acidic medium;

### DETAILED DESCRIPTION OF THE INVENTION

The present invention relates generally to the chemical reaction of a carbonyl group-containing compound, such as an aldehyde or ketone, with a primary or a secondary amine group to form a reversible imine or enamine. The imine or enamine may be useful in the treatment of allergic rhinitis in those instances where the primary or secondary amine group is part of a histamine moiety. In a preferred embodiment of the present invention, the resulting composition is buffered in an acidic medium.

In accordance with another aspect of the present invention, any natural or synthetic product that contains carbonyl groups can interact with histamine to form a reversible imine or enamine. For example, the present invention concerns the reaction of carbonyl-containing compounds such as aldehydes and ketones. Examples of aldehydes include, but are not necessarily limited to: retinal and pyridoxal-5-phosphate, which is also known as Vitamin B6. The resulting imine or enamine compounds generated from the reactions of the present invention are useful for the relief of histamine related ailments of the nasal passages, stomach, skin, eyes, mouth and throat. In another aspect, the present invention relates to the intranasal and oral administration of the novel inventive reaction product compositions for the relief of histamine-related issues.

In yet another aspect of the invention, any natural or synthetic product containing carbonyl groups can complex with biological amine compounds. Examples of naturally-containing amine compounds include, but are not necessarily limited to: serotonin; tysine; and various polyamines.

The compounds of the present invention thus provide novel pharmaceutical compositions for the treatment of allergic rhinitis comprising an imine, an enamine, or a combination of the foregoing. The present invention also relates to an isopropyl alcohol composition for treating symptoms of allergies by binding histamine with turmeric derivatives. The composition is buffered in a weak acidic medium to enable the reaction with histamine. By maintaining the composition in an acidic medium, the efficacy of the reactive components has been made very effective. Moreover, an acidic medium is preferred for the stability of turmeric solutions. The present invention thus provides novel pharmaceutical compositions for the treatment of allergic rhinitis, comprising:
a. a ketone, an aldehyde or a combination of the foregoing; and
b. optionally, an acid.

The present invention generally involves the reaction of ketones and aldehydes with histamines to provide novel compounds and methods for the treatment of allergic rhinitis. In one preferred embodiment, the interaction of curcumin with in vivo histamine in an acidic medium is presented. Compositions and methods of use are also described. The novel pharmaceutical compositions of the present invention result from the combination of:
a. a ketone, an aldehyde or a combination of the foregoing;
b. a primary amine, a secondary amine or a combination of the foregoing; and
c. optionally, an acidic medium.

A method for the treatment of allergic rhinitis in a human patient according to the present invention, therefore, comprises administering a pharmaceutically effective quantity of an aldehyde, a ketone, or a combination of the foregoing to the patient, optionally in acidic medium. The invention also comprises the administration of an imine, an enamine, or a combination of the foregoing to the human patient. More specifically, a method for treating allergic rhinitis in a human patient according to the present invention comprises administration of a pharmaceutically effective quantity of turmeric or a product formed from the reaction of histamine with a turmeric derivative to the human patient.

The chemical structure of histamine is identified as an imidazole ring having two carbon chains with a terminal amino group. Primary amines, which may be represented as RNH₂, undergo nucleophilic addition with aldehydes and ketones to yield imines, RₐR_{b}C≡NR Aldehydes and ketones, which both contain the carbonyl group, C═O, may be represented schematically as RCHO and RₐR_{b}C═O, respectively, where Rₐ and R_{b} may be the same or different. Similarly, secondary amines, represented as R_{c}R_{d}NH add aldehydes and ketones to produce enamines, R_{c}R_{d}NC═CRₐR_{b}, where RₐR_{b} and R_{c}R_{d} may be the same or different. Imines are important intermediates in several metabolic pathways. Imines and enamines are formed along with water in a reversible acid-catalyzed reaction process. This reversible condensation reaction makes imines very susceptible to hydrolysis and often difficult to isolate as pure substances. See: Organic Chemistry, L. G. Wade, Jr., 5^{th} Ed., Prentice Hall, New Jersey, 2003, p. 807-809.

One compound containing a carbonyl group is curcumin, a major component of the food spice turmeric. Curcumin, also known as diferuloylmethane, is known to have beta-diketone functionality in its molecule. A compound with a beta-diketone structure exists as a tautomer of keto and enol forms. While the structure of curcumin included herein is shown in the enol form, it will be understood by those knowledgeable in the relevant field that the keto form for the tautomer is also to be understood by implication.

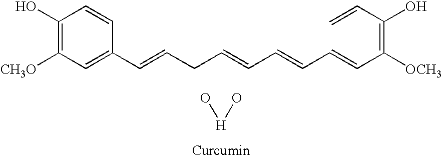

Turmeric has been used as a food additive, a medical agent, and a dye for cosmetics and fabrics without manifesting detrimental side effects for centuries. This record of safety may explain why the Food and Agricultural Organization and the World Health Organization expert committee on food additives approved curcuminoids as natural food coloring agents. With the acceptance of turmeric as a color additive for foods as safe (GRAS) by the FDA, the application of oleoresin, an extract of turmeric as a water-soluble yellow coloring agent in pickles, mustard, snacks, ice cream and beverages has found widespread use.

Turmeric (Curcuma longa), derived from the rhizome and root of the ginger-family, Zingiberaceae, is an ancient treasured spice used to flavor and color many traditional dishes. A traditional remedy in Ayurvedic, the ancient medical practice of the Indian system of medicine and Chinese medicine for over 5000 years, turmeric has been used through the ages as an “herbal aspirin” and “herbal cortisone” to treat a wide spectrum of infectious and autoimmune diseases. Indian Materia Medica, Popular Prakashan, 1976, a standard Ayurvedic reference, cites a large number of conditions where turmeric can be helpful as an adjunctive therapy, including systemic inflammations, microbial infections, skin lesions, blood disorders, and liver and stomach disorders. In Chinese traditional medicine, turmeric is known as “Jiang Huang” and is used to eliminate flatulence, resolve liver and urinary problems, for menstrual disorders, hemorrhaging, and fever and chest pain.

Although the chemical structure of this remarkable spice, food preservative and dye was identified by Lampe in 1910, it was only in the 1970s and 1980s that its many, varied health-promoting properties were identified. This was prompted by the discovery of the anti-oxidant properties of naturally occurring phenolic compounds. The capacity of phenolic compounds to trap and scavenge harmful oxygen radicals is an important protection mechanism in biological systems.

Curcuma longa has been found to contain a variety of phenolic compounds or curcuminoids. The three main curcuminoids are: curcumin, which is also known as diferuloylmethane or Curcumin-I as indicated above; demethoxy curcumin or Curcumin-II, which may also be written as p-hydroxycinnamoyl-feruloylmethane; and bis-demethoxy curcumin or Curcumin-III, which may also be written as p,p-dihydroxydicinnamoylmethane. Additional naturally-occurring curcuminoids, which may also be suitable for use in the inventive compositions contemplated herein, include Ar-turmerone, methylcurcumin and sodiumcurcuminate. See the structure of curcumin (I) above, and the structures of the remaining five compounds below. All six compounds exhibit a yellow color.

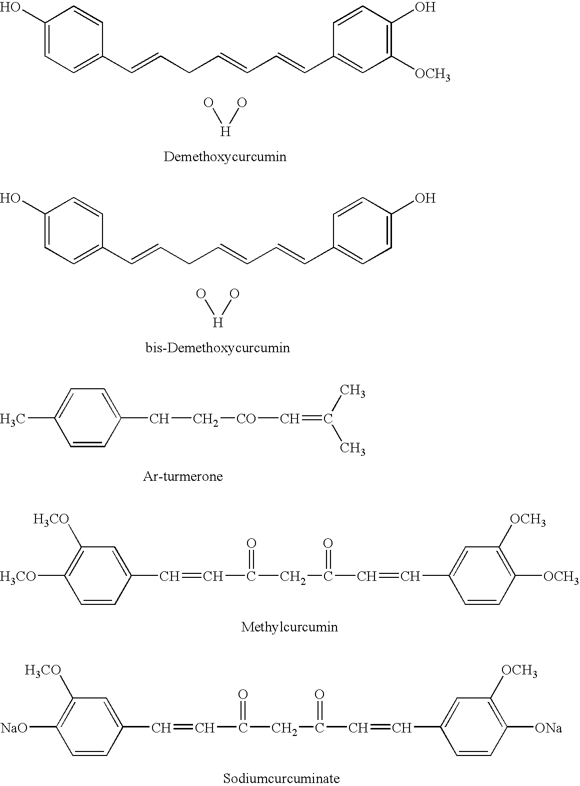

The pharmacological activities of curcuma longa and its constituents, which include anti-inflammation, anti-oxidation, anti-microbial, anti-tumor, anti-thrombic, anti-viral, anti-mutagen, anti-venom and hepatoprotective properties are cited in numerous research publications. Biological properties of curcumin emphasizing cellular and molecular mechanisms of action are summarized by Joe B., et al., Critical Reviews in Food Science and Nutrition, 2004; 44(2), pp. 97-111. Comprehensive reviews on the biological properties of curcuma longa are provided in Phytochemicals in Cancer Chemoprevention by Aggarwal, et al., August 2003, 1-24 and, Alternative Medicine Review, Araujo, et al., 2001, 96(5), 723-728. A list of over 80 U.S. institutions and laboratories that have published research work on turmeric and its components are available at the following internet address: http://www.newchapter.info/research/turmeric.html. Another website, www.curcuminoids.com provides an extensive review on the subject with more than 140 references.

As stated above, one aspect of the present invention concerns the chemical reaction of primary amines with carbonyl groups. Under proper conditions, a primary amine, RNH₂ reacts with aldehydes RₐCH═O and ketones RₐR_{b}C═O to yield imines RₐHC═NR and RₐR_{b}C═NR, respectively, where Rₐ and R_{b} may be the same or different. Secondary amines R_{c}R_{d}NH add similarly to yield enamines, R_{c}R_{d}NC═CCₐR_{b}, where R_{c} may not be the same as R_{d}.

A substituted imine with a C═N is also called a Schiff Base or an azomethine group. The mechanism of imine formation commences with a nucleophilic addition of the amine to the carbonyl group. This is followed by the protonation of the oxygen atom, and subsequent deprotonation of the nitrogen atom to give an unstable intermediate called a carbinolamine. A carbinolamine converts to an imine by losing water and forming a carbon-nitrogen double bond, C═N. This is considered a nitrogen analog of a carbonyl group.
RₐR_{b}C═O+RNH₂ [symbol] RₐR_{b}C═NR+H₂O Equation 1

Enamines are formed when an aldehyde or ketone reacts with a secondary amine, R_{c}R_{d}NH, again where R_{c} may or may not be the same as R_{d}. The process is similar to imine formation up to the iminium ion stage, but at this point there is no proton on nitrogen that can be lost to yield a neutral imine product. Instead, a proton is lost from the neighboring carbon yielding an enamine with a C═C bond. These reaction mechanisms are well documented in the literature.

As was found in the work towards the present invention, and will be appreciated is by those knowledgeable in the related field, proper pH is important to imine formation. The pH dependence of imine formation can be explained by inspecting each individual step in the mechanism. Since the second half of the amine-plus-ketone reaction is acid catalyzed, the solution must be somewhat acidic. If the solution is too acidic, however, the amine becomes protonated and non-nucleophilic, thus inhibiting the first step. It has been established that the reaction reaches a maximum rate at a weakly acidic pH of around 4 to 5. For example, the profile of pH vs. reaction rate observed for the reaction between acetone, a ketone, and hydroxylamine shows that the maximum reaction is obtained at a pH of 4.5. It should be noted that both imine and enamine formations are reversible and most of them can be hydrolyzed back to the original amine and corresponding ketone or aldehyde. See McMurray, J., Organic Chemistry, 5^{th} ed. Brook/Colole, Pacific Grove, Calif., 2000, pp. 770-774.

It should be emphasized that the initial binding of an amine to an aldehyde or ketone occurs during nucleophilic addition of the amine to the carbonyl group to form an unstable carbinolamine. Isolation of the unstable carbinolamine is not possible. However, an acid catalyst can be used to drive the reaction to form the imine or enamine product, which may be isolated. Thus, the use of an acid catalyst is not required for carbinolamine formation. Stated differently, the formation of an imine or enamine is not necessary for the inhibition of an amine. An amine may be inhibited through nucleophilic addition to a carbonyl group to form the carbinolamine intermediate.

Histamine, 2-(4-imidazolyl)-ethylamine is a hydrophilic molecule with an imidazole ring and an amino group connected by two methylene groups. It arises in vivo by decarboxylation of the amino acid, histadine. The molecule of histamine comprises a primary amine and a secondary amine group, which can be reacted with aldehyde or ketone groups to form imines and enamines, respectively, as described above.

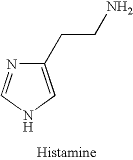

The structure of curcumin, the major component of Curcuma longa L. as diferuloylmethane was determined by Lampe et al., Ber. dtsch. chem. Ges. 1910, 43, 2163. The chemical structure was later confirmed by Roughley, et al., J. Chem. Soc., 1973, 20, 2379-2388. In the molecule of curcumin, the main chain is unsaturated and aliphatic, and the aryl group can be substituted. Heller, Ber. dtsch. chem. Ges., 1914, 47, 2998 reported that curcumin exists as a keto-enol structure. Srinivasan, Current Science, 1952, 311 isolated two new components of turmeric as demthoxycurcumin and bisdemethoxycurcumin. Three geometrical isomers of curcumin were also isolated. The three polyphenol pigments are collectively called curcuminoids and more commonly curcumin.

According to the present invention, and without being bound by theory, it is proposed that the ketone groups of the 1,3-diketone system present in Curcuma longa L. derivatives can interact with the primary and secondary amino groups of histamines in vivo to form reversible imines and enamines. The imines and enamines formed by the nucleophilic addition of primary and secondary histamine amines, respectively, to the carbonyl group of the curcuma β-diketone have been characterized in the course of the instant work by Fourier Transform infrared spectroscopy (FTIR). The infrared spectral data confirm the proposed mechanism. The present invention may thus be regarded as providing an in vivo pharmaceutical composition for the treatment of allergic rhinitis that comprises at least one of: a Schiff base, an imine, an enamine, as well as combinations of any of the foregoing. As indicated above, the pharmaceutical composition may, in addition, optionally include acidic medium. Alternatively, the present invention may be considered as providing an in vivo pharmaceutical composition resulting from combining:

a. a first moiety selected from among: an aldehyde; a ketone; and a combination of any of the foregoing;

b. a second moiety selected from among: a primary amine; a secondary amine; and a combination of any of the foregoing; and

c. optionally, acidic medium.

The present invention is further illustrated by the following examples.

### EXAMPLE 1

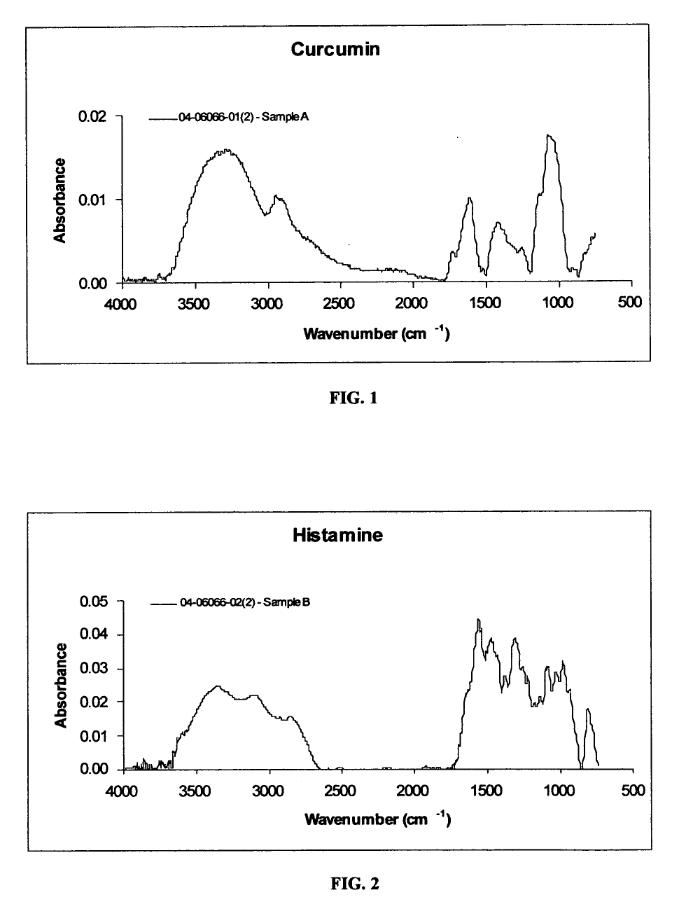

In Example 1, curcumin was obtained from Spectrum Chemical Company, Gardena, Calif. Histamine was procured from Aldrich Chemicals. The reported molecular weights of curcumin and histamine are 368 and 111, respectively. The FT IR spectra of curcumin and histamine are provided in FIG. 1 and FIG. 2, respectively.

As may be seen in FIG. 1, the infrared spectrum of curcumin exhibits: an O—H stretching absorption band at 3286 cm⁻¹; C—H stretching absorption bands at 2951 cm⁻¹ and 2916 cm⁻¹; a ketone carbonyl stretching absorption band at 1735 cm⁻¹; an enol carbonyl stretching absorption band at 1620 cm⁻¹; C—H bending absorption bands at 1431 cm⁻¹ and 1365 cm⁻¹; and C—O stretching absorption bands at 1261 cm⁻¹, 1145 cm⁻¹ and 1076 cm⁻¹. The foregoing is typical of a ketone-based material.

As shown in FIG. 2, the infrared spectrum of histamine exhibits: an N—H stretching absorption band at 3352 cm⁻¹; C—H stretching absorption bands at 2927 cm⁻¹ and 2856 cm⁻¹; a C—N stretching absorption band at 1561 cm⁻¹; C—H bending absorption bands at 1457 cm⁻¹, 1383 cm⁻¹ and 1313 cm⁻¹; and C—O stretching absorption bands at 1102 cm⁻¹, 1095 cm⁻¹ and 1034 cm⁻¹. The foregoing is typical of an amine-based material.

### EXAMPLE 2

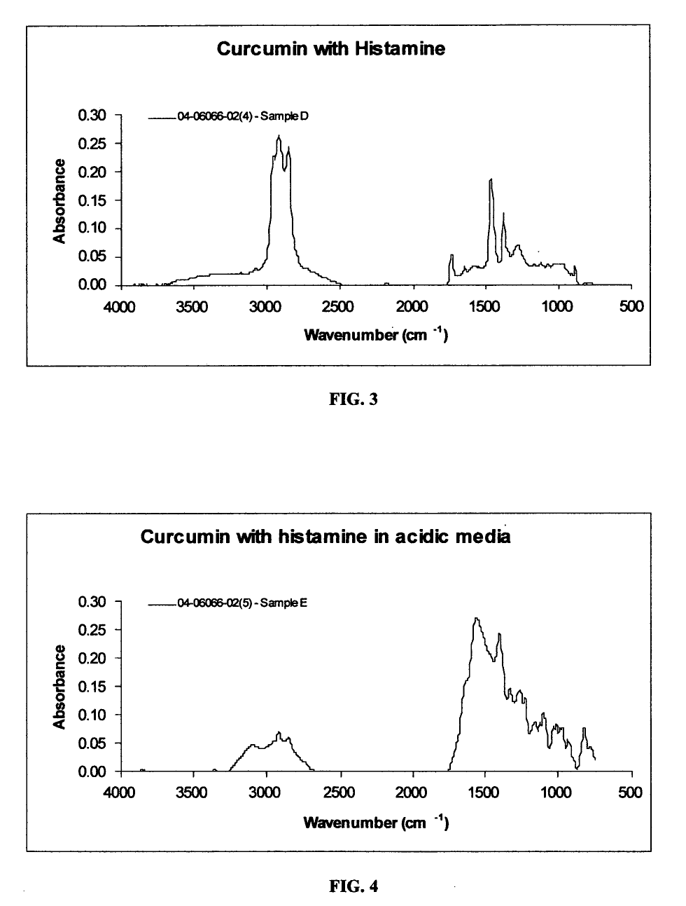

Curcumin is known to have very limited solubility in common solvents, including water. Due to its low solubility, solutions of curcumin and histamine were initially prepared at a 4:1 molar ratio of curcumin to histamine. In order to improve the solubility of the curcumin, its solution in isopropyl alcohol was warmed at 37° C. for 30 minutes and filtered. The histamine solution in isopropyl alcohol was then slowly added to the filtered curcumin solution under stir. The mixture was allowed to evaporate and dry at ambient conditions. The FT IR spectrum of the resultant product is provided in FIG. 3.

The infrared spectrum of the resultant product of curcumin and histamine in FIG. 3 exhibits an N—H absorption band as a broad shoulder around 3352 cm⁻¹, indicating that the product is not fully converted. The C—H stretching bands at 2955 cm⁻¹, 2922 cm⁻¹ and 2854 cm⁻¹; ketone carbonyl stretching band at 1735 cm⁻¹; and enol carbonyl stretching band at 1643 cm⁻¹ are exhibited in the spectrum. Many of the differences in the spectra arise from the intensity changes in the 1500 cm⁻¹ to 1800 cm⁻¹ region No new peaks indicating the presence of C—N stretching and C═C stretching modes are noticed in this region.

### EXAMPLE 3

Initial solutions of curcumin and histamine at 4 to 1 molar ratio were prepared in isopropyl alcohol. Drops of glacial acetic acid in the form of a 10% solution were added to the curcumin solution to adjust the pH to between 4 and 5. The solution was then warmed at 37° C. for 30 minutes and filtered. The histamine solution was gradually added to the clear curcumin solution under slow agitation. The resultant composition was allowed to dry at ambient conditions. An infrared spectrum of the resultant product is given in FIG. 4. Examples of acids which may be used according to the teaching of the present invention, in addition to acetic acid used in Example 3, include, but are not necessarily limited to: ascorbic acid, citric acid and acetic acid, taken alone or in combination with any of the foregoing.

The infrared spectrum of the 4 to 1 mixture of curcumin and histamine prepared according to Example 3 shown in FIG. 4 exhibits: an O—H stretching absorption band at 3100 cm⁻¹; and C—H stretching absorption bands at 2955 cm⁻¹, 2922 cm⁻¹ and 2853 cm⁻¹. The spectrum shows that the N—H stretching absorption band of histamine is absent in this region, thereby suggesting that the histamine reacted with the carbonyl group of curcumin. The C═O stretching frequency of curcumin at 1735 cm⁻¹ has completely disappeared, demonstrating that the reactants are fully converted. The absorption bands of imine C═N and enamine C═C are known to exhibit in the range of 1550 cm⁻¹-1700 cm⁻¹. The spectrum in FIG. 4 shows a very strong absorption at 1562 cm⁻¹, indicating the formation of an imine.

### EXAMPLE 4

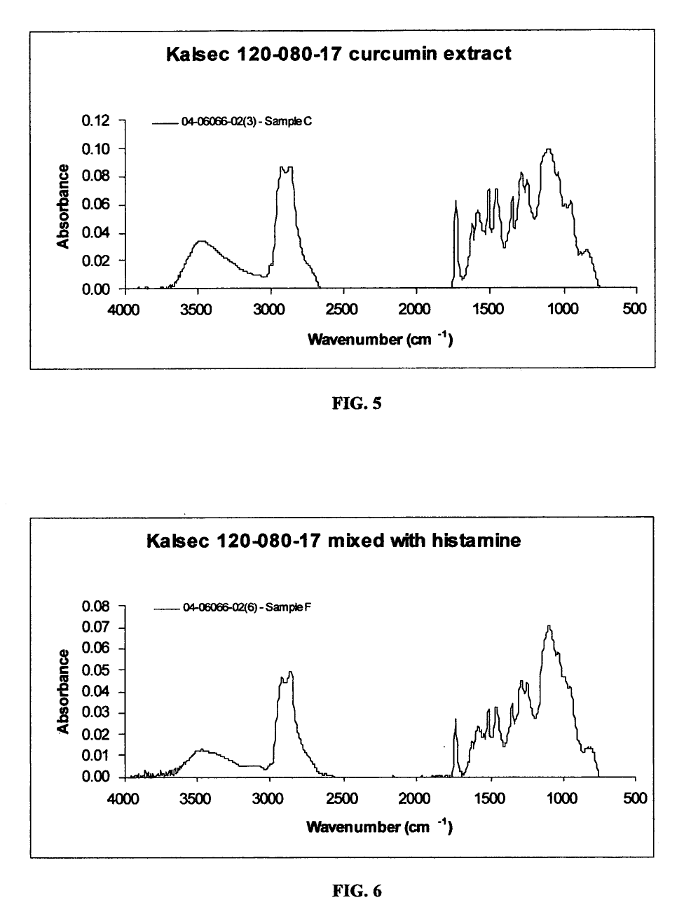

A sample of curcumin extract from Kalsec Corporation was used in studying Example 4. The water-soluble Kalsec 120-080-17 containing 8% curcumin and 92% Polysorbate 80 as described above was mixed with histamine in 2 to 1 molar ratio. To an isopropyl alcohol solution of 120-080-17, an isopropyl alcohol solution of histamine was added under gentle agitation at room temperature. The resultant composition, which exhibited a dark red color and a syrupy consistency, was allowed to dry at ambient conditions. This syrupy concentrate was used for infrared spectral analysis. The infrared spectrum of Kalsec 120-080-17 provided in FIG. 5 exhibits: an O—H stretching absorption band at 3485 cm⁻¹; C—H stretching absorption bands at 2924 cm⁻¹ and 2863 cm⁻¹; a ketone carbonyl stretching absorption band at 1735 cm⁻¹; C—H bending absorption bands at 1625 cm⁻¹, 1586 cm⁻¹, 1513 cm⁻¹, 1450 cm⁻¹ and 1350 cm⁻¹; and C—O stretching absorption bands at 1286 cm⁻¹, 1248 cm⁻¹ and 1100 cm⁻¹.

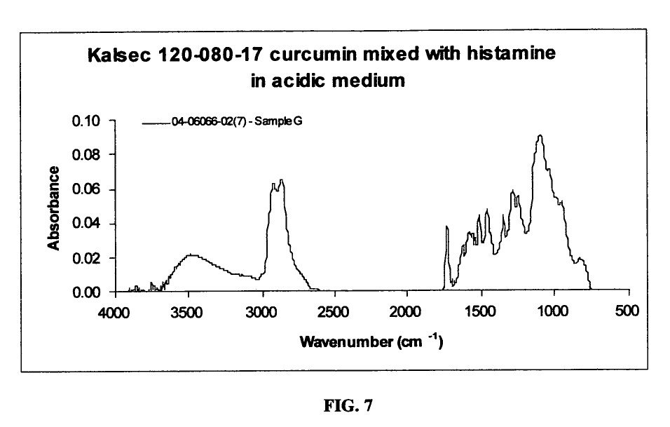

The infrared spectrum of Kalsec 120-080-17 curcumin extract mixed with histamine showed no significant change in the spectral bands as shown in FIG. 6, suggesting that the curcumin in Polysorbate did not undergo condensation with histamine.

### EXAMPLE 5

In this example, the Kalsec 120-080-17 curcumin dispersion in isopropyl alcohol was acidified with glacial acetic acid to a pH of 4 to 5. Histamine solution in isopropyl alcohol was then added to the acidified curcumin dispersion at room temperature to yield an acidified histanine-curcumin composition. The molar ratio of curcumin to histamine was 2 to 1. The resultant orange red color solution was dried at ambient conditions. The syrupy concentrate was used for the FT IR analysis as shown in FIG. 7. The infrared spectrum of the acidified mixture of curcumin extract with histamine indicated no significant changes in the spectral bands.

The infrared data presented above indicate that curcumin binds with histamine in an acidic medium. The data also suggest that the curcumin extract in Polysorbate 80 when mixed with histamine was not effective in a condensation reaction. The present invention, therefore, indicates that curcumin can be employed successfully for therapeutic management of allergic disorders by controlling or blocking histamine. Derivatives of curcuminoids that can be included for use according to the present invention include, but are not necessarily limited to acid adducts such as: HCl, SO₄, acetate, and oxalate, either alone or in combination. While providing the acidity this may also improve the solubility of curcuminoids.

Additional curcuminoids or analogs that can be used according to the teaching of the present invention are curcuminoids that include substituents in the structure to provide or enhance acidity. Examples of substituents of curcuminoid that may be used in accordance with the present invention include, but are not necessarily limited to: —OH, —NO₂, —SO₃, —OCH₃, methyl, ethyl, n-propyl, isopropyl, as well as combinations of any of the foregoing. As will be appreciated by those knowledgeable in the relevant field, one or more substituents on any of the curcumin ring positions may be possible, provided that such substitutions do not negatively impact the reactivity of the curcuminoid carbonyl.

Also within the scope of the present invention are substituted derivatives of curcumin or curcuminoids in which the hydroxyl group, commonly written —OH, is replaced by a metal atom. Suitable metal atoms for use in substituted curcuminoids include, but are not necessarily limited to: sodium, potassium, calcium, as well as combinations of any of the foregoing.

### EXAMPLE 6

In this Example, acid buffers of curcumin, curcuminoids or substituted curcuminoids may be combined with histamine in accordance with the teaching of the present invention. Acid buffers are mixtures of weak acids and their conjugate bases, and enable better control of the pH of the resulting solution. An example of one buffer system contemplated for use with the present invention would be a mixture of citric acid and sodium citrate.

### EXAMPLE 7

The present invention also contemplates that curcumin can be administered in dry form in the absence of an acid. In one embodiment of the present invention, dry curcumin can be blended with pharmaceutical grade inert ingredients to be administered to a patient in the form of a tablet or capsule. According to another embodiment of the present invention, curcumin can be mixed with acids such as citric and ascorbic acid and packaged in the form of tablets or capsules. According to a preferred embodiment of the present invention, the mixture is present in the form of an acidic medium when it comes into contact with the histamine in order for the above-described condensation reaction to occur.

### EXAMPLE 8

Another class of compounds that can be used to interact with histamine according to the teachings of the present invention includes the class composed of compounds containing carbonyl groups found in naturally occurring aldehydes and ketones such as, but not necessarily limited to: cinnamaldehyde, citral (lemon grass), vanillin, carvone, camphor, zingerone, feverfew (a sesquiterpene lactone, 4,5β-epoxy-germacra-1-(10), 11-(13)-dien-12,6-olide (parthenolide), isolated from Tanacetum parthenium) and carbohydrates. Additional carbonyl-group containing compounds, which may be used in accordance with the present invention include those having animal origins. The latter include, but are not necessarily limited to: testosterone, progesterone, muscone and cortisone. The derivatives of these compounds include structural modifications as described above wherein portions of the molecule may be changed via substitution, provided that the functional group is not altered in any way.

Isolation of substituted imines, which are also known as Schiff Base compounds as indicated above, by the interaction of citraldehyde, citral, camphor and vanillin with histamine are described in the Examples 9 through 12, respectively.

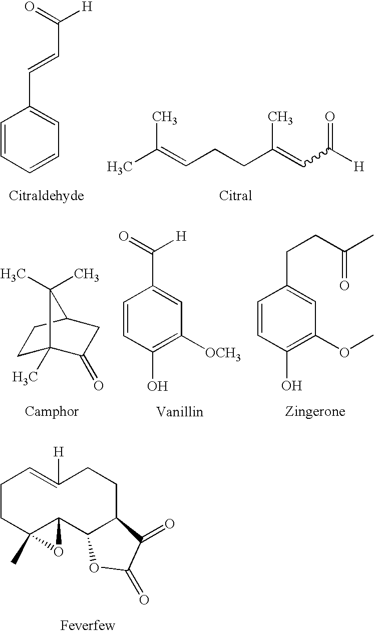

### EXAMPLE 9

In Example 9, citraldehyde, with a molecular weight of 132.16, was obtained from Sigma Aldrich Chemicals. To 2 moles of citraldehyde in isopropyl alcohol was added a few drops of water acidified with acetic acid to achieve a pH of around 4. One mole of histamine in isopropyl alcohol was added to the citraldehyde solution under slow agitation. The solution was warmed at 37° C. for 30 minutes, followed by the addition of crushed ice. A yellow precipitate separated out, indicating the formation of a Schiff Base compound.

### EXAMPLE 10

Citral, commonly known as lemon grass, was procured from Sigma Aldrich Chemicals. The molecular weight of the liquid compound is 152.13. Isopropanol solutions of citral and histamine were mixed in 2 to 1 molar ratio. Prior to the addition of histamine, a few drops of acidified water were added to the citral solution as described in EXAMPLE 9 above. The resulting solution for Example 10 was warmed at 37° C. for 30 minutes. Addition of crushed pieces of ice to the hot solution enabled the separation of a yellow Schiff base product, suggesting the condensation of the citral carbonyl group with histamine.

### EXAMPLE 11

Camphor used in Example 11 was obtained from Sigma Aldrich Chemicals. The reported molecular weight of camphor is 152.23. A histamine solution in isopropyl alcohol was added to an acidified aqueous isopropanol solution of camphor in 1:2 molar ratio (histamine:camphor). After warming the resulting mixture to 37° C. for 30 minutes, the solution was chilled using pieces of ice. An off-white precipitate separated out, suggesting the presence of an azomethine group in the product.

### EXAMPLE 12

Vanillin, with a molecular weight of 152.15, was purchased from Sigma Aldrich Chemicals. Isopropanol solutions of vanillin and histamine were mixed at 2:1 molar ratios. The vanillin solutions were acidified prior to adding the histamine as described in Example 9 above. The resulting solutions were warmed to 37° C. for 30 minutes. Addition of ice to the solutions separated a yellowish brown liquid, indicating the formation of Schiff Base compounds.

### EXAMPLE 13

In yet another embodiment of the present invention, curcumin, curcuminoids, substituted derivatives of the foregoing, cinnamaldehyde, citral, camphor and vanillin can interact with a large number of medically and biologically significant amine compounds in addition to histamine. Examples of other amines that may be also suitable for use with the present invention include, but are not necessarily limited to: serotonin, tysine, and polyamine, as well as combinations of any of the foregoing. Also within the scope of the present invention is the use of carbonyl group-containing compounds to interact with biological amine compounds. The process can be enhanced in an acidic medium.

### EXAMPLE 14

Another aspect of the present invention is the use of curcumin, curcuminoids, substituted curcumins, cinnamaldehyde, citral, camphor and vanillin in combination with one or more additional pharmaceutical compounds such as decongestants, cough suppressants, H1 and H2 histamine blockers, anti-inflammatory agents, etc., to provide mixtures offering synergistic effects. These mixtures can be administered in a combined formulation or separately but simultaneously with curcumin, curcuminoids, curcumin derivatives, cinnamaldehyde, citral, camphor or vanillin. The formulations containing curcumin, cinnamaldehyde, citral, camphor and vanillin can be administered in conventional tablet, capsule, gel, liquid, spray or injection form using pharmaceutically acceptable carriers, fillers, excipients, preservatives and other ingredients required to meet specific applications as will be recognized by those knowledgeable in the relevant arts. These compounds can also used as supplements in food and beverage products.

The present invention has been described above in detail with respect to specific embodiments and figures, and examples demonstrating the successful reduction to practice have been provided. These embodiments should not be construed as narrowing the scope of the invention, but rather as illustrative embodiments. It is to be further understood that various combinations, modifications and substitutions may be made by those knowledgeable in the relevant art to the described starting materials, reactions and inventive compositions described herein as well as to methods of treatment and use of the inventive compositions, without departing from the broad scope of the invention as intended to be described herein. The scope of the invention is further defined by the following claims.

What is claimed:

1. A pharmaceutical composition for the inhibition of histamine, comprising:
a. a first moiety comprising a ketone, an aldehyde or a combination of the foregoing; and
b. optionally, an acidic medium.

2. The pharmaceutical composition of claim 1, wherein the first moiety is characterized as a curcuminoid, cinnamaldehyde, citral, camphor, vanillin, carvone, camphor, feverfew, zingerone, substituted forms thereof, as well as combinations of any of the foregoing.

3. The pharmaceutical composition of claim 2, wherein the curcuminoid is selected from among: diferuloylmethane; p-hydroxycinnamoyl-feruloyhnethane; p,p-dihydroxydicinnamoylmethane, Ar-turmerone, methylcurcumin, sodiumcurcuminate and tetrahydrocurcumin; as well as combinations of any of the foregoing.

4. A method for the treatment of allergic rhinitis, comprising inhibiting a histamine molecule.

5. The method of claim 4, wherein the inhibiting comprises reversibly forming a moiety selected from among a Schiff base, an imine, an enamine, as well as a combination of any of the foregoing, upon combination of a carbonyl group-containing compound with the histamine.

6. The method of claim 5, wherein the carbonyl group is selected from among aldehydes, ketones, as well as combinations of any of the foregoing.

7. The method of claim 5, wherein a source for the carbonyl group is a molecule selected from among the group including curcumin and curcuminoids, cinnamaldehyde, citral, camphor, vanillin, carvone, camphor, feverfew, zingerone, including substituted forms thereof, as well as combinations of the foregoing.

8. The method of claim 7, wherein the curcuminoid molecule is selected from among: diferuloylmethane, p-hydroxycinnamoyl-feruloylmethane; p,p-dihydroxydicinnamoylmethane, Ar-turmerone, methylcurcumin, sodiumcurcuminate and tetrahydocurcumin; as well as combinations of any of the foregoing.

9. The method of claim 5, wherein the carbonyl-group containing compound optionally comprises acidic media.

10. The method of claim 5, wherein a pharmaceutically effective quantity of the carbonyl-containing compound is administered to a human patient.

11. An in vivo pharmaceutical composition for the treatment of allergic rhinitis, comprising at least one of: a Schiff base, an imine, an enamine, as well as combinations of any of the foregoing; and optionally comprising acidic media.

12. The in vivo pharmaceutical composition of claim 11, resulting from combining:
a. a first moiety selected from among: an aldehyde; a ketone; and a combination of any of the foregoing;
b. a second moiety selected from among: a primary amine; a secondary amine; and a combination of any of the foregoing; and
c. optionally, acidic medium.

13. A pharmaceutical composition for the treatment of allergic rhinitis, comprising the combination of:
a. a first moiety comprising a ketone, an aldehyde or a combination of the foregoing;
b. a second moiety comprising a primary amine, a secondary amine or a combination of the foregoing; and
c. optionally, an acidic medium.

14. A method for the treatment of allergic rhinitis in a human patient, comprising administering a pharmaceutically effective quantity of a carbonyl-containing compound.

15. The method of claim 14, wherein the carbonyl-containing compound is selected from among: curcumin or a curcuminoid, cinnamaldehyde, citral, camphor, vanillin, carvone, camphor, feverfew, zingerone, as well as substituted forms thereof, and combinations of any of the foregoing.

16. A method for the treatment of allergic rhinitis in a human patient, comprising:
a. administering a pharmaceutically effective quantity of a first moiety to the patient, the first moiety selected from among: a first Schiff base, a first imine, a first enamine as well as combinations of any of the foregoing;
b. hydrolyzing the first moiety in vivo to yield a carbonyl group-containing compound; and
c. forming a second moiety upon reaction of the carbonyl group-containing compound with in vivo histamine, the second moiety characterized as: a second Schiff base, a second imine, a second enamine as well as combinations of any of the foregoing.

17. The method of claim 16, wherein the carbonyl group-containing compound is characterized as a curcuminoid, cinnamaldehyde, citral, camphor, vanillin, carvone, camphor, feverfew, zingerone, substituted forms thereof, as well as combinations of any of the foregoing.

18. The method of claim 17, wherein the curcuminoid is selected from among: diferuloylmethane; p-hydroxycinnamnoyl-feruloylmethand; p,p-dihydroxydicinnamoylmethane, Ar-turmerone, methylcurcumin, tetrahydocurcumin and sodiumcurcuminate; as well as combinations of any of the foregoing.
